(19) United States
(12) Patent Application Publication
(10) Pub. No.: US 2024/0033397 A1
(43) Pub. Date: Feb. 1, 2024

(54) BIOACTIVE NANO FIBRE DRESSING FOR TREATING WOUNDS AND A METHOD FOR PREPARATION THEREOF

(71) Applicants: Pranay Wal, Kanpur (IN); Ravinesh Mishra, Mohali (IN); Bhupen Kalita, Guwahati (IN); Chinmaya Mahapatra, Koraput (IN); Hemanta Pathak, Barpeta (IN); Sumon Giri, Howrah (IN); Devi Violina, Sonitput (IN); Joyanta Kishore Debnath, Morigaon (IN); Namrata Koushik, Sonitpur (IN); Manish Bhise, Akola (IN); Mohd Imran, Rafha (SA)

(72) Inventors: Pranay Wal, Kanpur (IN); Ravinesh Mishra, Mohali (IN); Bhupen Kalita, Guwahati (IN); Chinmaya Mahapatra, Koraput (IN); Hemanta Pathak, Barpeta (IN); Sumon Giri, Howrah (IN); Devi Violina, Sonitput (IN); Joyanta Kishore Debnath, Morigaon (IN); Namrata Koushik, Sonitpur (IN); Manish Bhise, Akola (IN); Mohd Imran, Rafha (SA)

(21) Appl. No.: 18/481,143

(22) Filed: Oct. 4, 2023

(51) Int. Cl. A61L 15/40

(52) U.S. Cl. CPC A61L 15/40; A61L 2400/12

(57) ABSTRACT

The present disclosure relates to a herbal bioactive dressing for healing wounds and a method thereof. The dressing includes an outer layer having a matrix of nanofibre formed by mixing cow urine with extracts obtained from Aloe Vera, Calandula, Curcumin, Sandal wood in same amount and a self-adhesive inner layer. The dressing incorporated into the nanofiber patches through electrospinning.

### TECHNICAL FIELD

The present application relates to treatment for wounds. More particularly, the present application discloses a bioactive nano fibre dressing for treating wounds and a method for preparation thereof.

### BACKGROUND

The wound healing is a complex but highly dynamic process of cellular, physiological and biochemical events, which leads to the functional restoration of injured tissue. Cowpathy is a treatment based on products obtained from Indigenous cow as used in Ayurveda. The ancient Ayurvedic literature (VirCharak Samhita, Sushrut Samhita, Gad Nigrah) suggests a number of pharmacological applications of Prime Products from Indigenous cows such as in treatment of leukoderma, hyperlipidemia, arthritis, renal disorders, dietary disorders, gastro-intestinal tract disorders, acidity, diarrhea, dysentery, Cancer, diabetes, blood pressure, asthma, psoriasis, eczema, ringworm, itching, heart attack, blockage in arteries, fits, gynecological problems, ear and nose problems, etc.

In ancient Indian Ayurvedic literature such as Charak Samhita and Sushruta Samhita, cow urine is mentioned as the most effective substance of animal origin with innumerable therapeutic value. Though many Vaidyas and practitioners are using Prime Products from Indigenous cows for various treatments, there is not much scientific studies and research on the same. Cow urine and its distillate is being used by Ayurveda practitioners throughout the country.

Medicinal properties of cow urine such as bioenhancer, antibiotic, antifungal, and anticancer have been patented under U.S. Pat. Nos. 6,896,907 and 6,410,059. As cow urine has shown wound healing properties in various studies done in india and abroad, so we have developed an innovative bioactive dressing (Herbal patch) for treating wounds using cow urine.

According to current estimations around 6 million people suffer from conditions of chronic wounds worldwide but reports for Indian population suggest that 4.5 per 1000 people population suffer from chronic and 10.5 per 1000 people suffer from acute wounds.

The process of healing is complex and is dependent on many factors who work to restore the injured tissue. If the healing does not progress in a normal manner then a chronic wound may become a problem to the patient. It has been estimated that cost of medically maintaining a diabetic ulcer is around US$50,000 while that of a chronic ulcer is US$25 billion yearly. With the number of patients increasing yearly (6.5 million per year) and dominating prevalence of lifestyle disease conditions like diabetes and chronic conditions which affect wound healing. Vasconez et al. explored latest advancements spanning several facets of wound healing, including biologics, skin substitutes, biomembranes and scaffolds. He suggested that healing of surface and deep wounds of the epidermis is a complex multistage process, but one that may nevertheless be expedited utilizing strategies such as the application of active biologic, biomembrane or scaffold based wound dressings.

Naveen et al. carried out a comparative study showed that sustained-release silver foam dressing has faster wound healing, lesser pain with earlier hospital discharge than 1% silver sulfadiazine and lesser expenses when total hospital burden was accounted.

Nanofibers have significant potential in the targeted delivery of drugs. The high surface-area-to-volume ratio in nanofiber scaffolds allows increased exposure of molecular chains to the nanofibers, creating more opportunities for binding and catalytic reactions. In recent years, the health concerns associated with the side effects of synthetic compounds used in cosmetics, medicine, and food industry and the emergence of antibiotic resistance of pathogens have driven electro spinning research towards the development of fibres encapsulating plant extracts. In fact, plants are sources of many chemical compounds that exhibit antimicrobial activity and have the potential to treat antimicrobial-resistant infections and also anti-inflammatory properties.

### SUMMARY

In an aspect of the present disclosure, a herbal bioactive dressing for healing wounds is disclosed. The dressing includes an outer layer comprising a matrix of nanofibre formed by mixing cow urine with extracts obtained from Aloe Vera, Calandula, Curcumin, Sandal wood in same amount and a self-adhesive inner layer.

In an aspect, the dressing is incorporated into the nanofiber patches through electrospinning.

In an aspect, the matrix is non-absorptive and non-dissolving.

In another aspect, a method for preparation of herbal bioactive dressing for healing wounds is disclosed. The method involves isolating extracts from a plurality of herbs comprising Aloe Vera, Calandula, Curcumin, Sandal wood, followed by collecting cow urine at different interval from five different indigenous breeds of cows. The method involves performing comparative assessment of the herbs with cow urine in terms of physical stability, organoleptic parameters and chemical instability, followed by mixing the cow urine with the extracts at 40-45 deg C. by maceration process for 72 hours to form a cow urine extract and filtering the cow urine extract to obtain the filtrate. The method involves concentrating the filtrate to a semi solid mass by using vacuum distillation apparatus and incorporating the semi solid mass into a nanofiber patch.

In an aspect, the maceration process involves pouring at least 25 grams of each of the herbs into at least 150 cc absolute ethanol, followed by placing the mixture in a shaking incubator at 30 deg C. and at 100 rpm for 48 hours. The method of claim 7, wherein the maceration process involves evaporating the ethanolic extract using rotary evaporator for at least 25 min to obtain pasty extract.

One should appreciate that although the present disclosure has been explained with respect to a defined set of functional modules, any other module or set of modules can be added/deleted/modified/combined, and any such changes in the architecture/construction of the proposed system are completely within the scope of the present disclosure. Each module can also be fragmented into one or more functional sub-modules, all of which are also completely within the scope of the present disclosure.

### BRIEF DESCRIPTION OF DRAWINGS

The accompanying figures (Figs.) illustrate embodiments and serve to explain the principles of the disclosed embodiments. It is to be understood, however, that these figures are presented for purposes of illustration only and not for defining the limits of relevant applications.

FIG. 1 illustrates various pictures for a process for healing wound, in accordance with embodiments of the present disclosure;

FIG. 2 illustrates a flowchart depicting steps for preparation of a dressing for healing wound, in accordance with embodiments of the present disclosure;

### TABLE

Table 01 represents histological changes with comparison of our bioactive dressing with standard bandage and antiseptic cream used for minor wounds, in accordance with embodiments of the present disclosure.

### DETAILED DESCRIPTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein and any additional applications of the principles of the invention as illustrated herein, which would occur to one skilled in the relevant art and having possession of this disclosure, are to be considered within the scope of the invention.

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Various terms, as used herein, are shown below. To the extent a term used in a claim is not defined below, it should be given the broadest definition persons in the pertinent art have given that term as reflected in printed publications and issued patents at the time of filing.

In an embodiment of the present disclosure, a herbal bioactive dressing for healing wounds is disclosed. The dressing includes an outer layer which has a matrix of nanofibre formed by mixing cow urine with extracts obtained from Aloe Vera, Calandula, Curcumin, Sandal wood. All the herbs are mixed in same amount. The dressing has an inner layer which is self-adhesive. The matrix is non-absorptive and non-dissolving.

### Method of Preparation

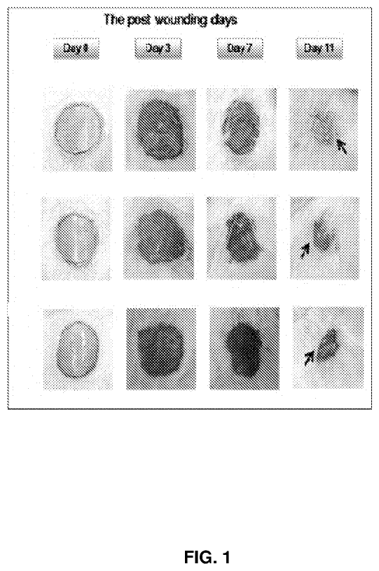

Herbal extracts and excipients were weighed separately and in combination of extracts, pure excipients, Herbal extracts/excipients samples in 1:1 ratio. Individual herbal extracts, individual excipients and Herbal extracts/excipient combinations was transferred in to an appropriately labelled glass vial. Subsequently, 10 μL of ultra pure water was added to each vial and mixed using a glass capillary. Each vial was sealed properly and placed in hot air oven at 50° C. for 4 weeks. To identify the physical instability, organoleptic parameters of samples such as color and texture were observed initially and at the end of 1st, 2nd, 3rd and 4th week and to identify the chemical instability, samples were used to record the Fourier-Transform Infrared (FT-IR) spectrum using FT-IR Spectrometer. Disappearance of absorption bands or reduction of the band intensity combined with the appearance of new bands give clear evidence for interactions. Process of wound healing as shown in FIG. 1.

### Drug Identification Tests

### Melting Point Determination

Melting point of herbal extracts was determined by Differential Scanning calorimetry (DSC) from CDRI, Lucknow, India.

### Solubility Studies of Herbal Extracts

Semi quantitative determination of the solubility was made by adding solvent to volumetric flask containing accurately weighed amount of individual extracts. The system was shaken vigorously and examined visually for any undissolved solute particles. The solubility was expressed in terms of ratio of solute and solvent. The solubility study of extracts was performed in different solvents like methanol, ethanol, ether, distilled water, mixture of methanol and phosphate buffer (60:40, 70:30, 80:20) and Phosphate buffer solution pH 7.4, 5.5, 6.8 separately by keeping the on-vortex mixture.

Determination of Absorption Maxima (λmax) for Different Herbal Extracts by High Pressure Liquid Chromatography (HPLC)

### Preparation of Stock Solution and Working Solution:

Stock solution and working solution of individual extracts was prepared for scanning of maximum absorption by HPLC as well as method development studies were performed in order to get highest absorption of individual drugs in specific solvent or ratio of solvent: co-solvent.

### Preparation of Calibration Curve

Calibration curve was prepared according to Beer's Lamberts range of concentrations of individual herbal extracts.

### Validation of Calibration Curve

Validation of curve was performed on basis of Linear Regression Analysis using MS-Excel 2016 and r2 value will reveal the validation of calibration curve.

### Determination of Partition Co-Efficient of Different Herbal Extracts

Required amount of different extracts was shaken individually for 30 minutes in separating funnel with 20 ml (1:1) mixture of n-octanol and water. Both the aqueous layer and oil layer was separated out and the amount of drug dissolved in aqueous phase and oil phase was determined by using HPLC.

### Preparation of Herbal, Extracts

Maceration method was used for extraction. Twenty-five grams of selected herbs were poured into 150 cc absolute ethanol (Merck, Germany), and then, the mixture was placed in the shaking incubator at 300 C and at 100 rpm for 48 h. Afterwards the ethanolic extract was evaporated using rotary evaporator for 25 min to obtain pasty extract.

### Preparation of Polymeric Solution

### Electro Spinning Polyurethane (PU) Solution

For preparation of polymer solution, the polymer mixture of PU: Collagen at three different levels (30:70, 40:60 and 50:50% v/v) was dissolved in dichloromethane with concentration of 10% w/v. The required amounts of herbs were added into PU/Collagen solution to obtain electrospinning solution and mixed together at 60° C. for 15 min.

### Electrospinning Poly ε-Caprolactone Solution

Poly ε-caprolactone (PCL) with 80,000 (g mol-1) molecular weight and collagen (30:70, 40:60 and 50:50% v/v) was dissolved in determined concentration of chloroform: DMF (70:30% v/v). The required amounts of herbs were added into prepared polymeric solution and mixed together at 60° C. for 15 min.

### Preparation, Antimicrobial and Release Behaviors of Nisin-Poly (Vinyl Alcohol)/Wheat Gluten/ZrO2 Nanofibrous Membranes

Modification of PCL Electrospun Nanofibrous Mat with Herbal Extract for Improved Interaction with Cells

### Characterization of the Polymeric Solutions

The apparent viscosity of the polymeric solutions was determined using Brookfield viscometer at different shear rates. The conductivity of the solutions was measured using a conductivity meter. All measurements were performed in triplicate at 25° C. The bioactive dressing has a longer time on the site of action, because of less chances of rubbing with clothes.

After detailed study of the healing properties of cow urine and plant extracts. the extracts from plants were extracted. Such extracts are useful in treating wounds. Then, the compatibility studies of various herbs like Aloe Vera, Calandula, Curcumin, Sandal wood in cow urine were performed.

Then Comparative assessment of various herbs with cow urine in terms of

physical stability, organoleptic parameters etc and for chemical instability, samples were used to record the Fourier-Transform Infrared (FT-IR) spectrum was performed.

Then the final stable extract was incorporated in the nano fiber patches through the electrospinning machine. The specifications of the present invention patch include two layers an outer layer which is made up of a non-absorptive and non-dissolving matrix of nanofibre formed by mixing cow urine with extracts of Aloe Vera, Curcumin, Sandal wood etc. The inner layer was self-adhesive hence it protected underlying tissues by forming a layer thus sealing the wound region.

### Experimental Details

Wistar albino rats of either sex with an approximate weight of 200-350 g were placed in individual polypropylene cages and acclimatized to the laboratory environment for 1 week. The rats were maintained on normal laboratory food and water ad libitum, under controlled room temperature (25° C.±2° C., 60-70% humidity) and 12-12 h light-dark cycle, so after preparing the proposed dressing we initiated the pre-clinical studies by examining the wound healing process on Control, Treated and Non-Treated groups for better evaluation of the results. (The treatment with herbal patch was compared with other treatment using ointment and gels, for this a minor incision was done on the shaved skin of animal and then it was stitched and later the herbal patch was applied in one group comparative to other treatments available in another group.

All animal experiments were carried out according to the ethical guidelines as established by Animal ethical committee of our college, which is approved by CPCSEA till 2022. As wound animal model, Male rats, having the weight of 270-280 g, were selected. Briefly, the dorsal hair was shaved and decontaminated with 70% ethanol after anesthetization, and then the electric device heated at 850 C was contacted with the shaved dorsal skin for 15 sec. The burn-wound area was cooled with an ice pack after sedation. After 24 h, the full thickness of dead epidermis layer around the wound area was excise by using a sterilized curette. Three rats were used for each group, such as gauze, NF and electrospun optimized nanofiber, per time period (1, 2, and 3 weeks). Each rat was having four wounds on the dorsal skin, and they were covered with gauze, NF and electrospun optimized nanofiber with 1.5 cm diameter, respectively. After applying each sample on the burn-wounds, adhesive bands were wrapped around the burn wounds (Soft cloth tape). The wound dressings were replaced by new ones every third day. At the same time, the body weights of all rats were accurately measure.

The process of burn-wound closure was observed by using a digital camera every third day after treatment. Margin of the wound area was traced onto the wound-photograph by using 10.0 Adobe Photoshop CS3 Extended software. The rate of burn-wound closure was calculated as follows:

Wound reduction (%)=[(Ao−At)/Ao]×100

where Ao and At represent the initial wound area and the wound area after a time interval.

The tissue samples containing part of the wound area were taken for histological analysis. The tissues were fixed in 4% formaldehyde, and dehydrated through graded alcohol series. After embedding in paraffin, the samples were cut to a 6 μm thickness and stained with hematoxylin and eosin (H and E). The harvested tissues from each group were lysed by the addition of cold RIPA lysis buffer containing 0.5 M Tris-HCl (pH 7.4), 1.5 M NaCl, 2.5% deoxycholic acid, 10% NP-40 and 10 mM EDTA with protease and phosphatase inhibitors. The tissue lysates were e incubated in ice for 30 min and then centrifuged at 1.3×104 rpm for 10 min. The concentration of protein was measured using a micro-plate spectrophotometer at a wavelength of 595 nm. An equal amount of protein (40 μg) was heated for 10 min at 100 OC and subjected to sodium dodecyl sulfate polyacrylamide gel electrophoresis (SDS-PAGE). Then the protein were transferred onto a nitrocellulose transfer membrane for 90 min. After blocking with 5% non-fat dry milk, the membrane was proved overnight with a primary antibody (Mouse anti-rat MMP-2 or Rabbit antirat MMP-9) followed by incubation with a respective secondary antibody conjugated to horseradish peroxidase.

### Method for Developing the Bioactive Dressing

The cow urine (Gaumutra) was collected at different time interval from 5 different indigenous breeds of cow as sample. The cows used was on standard diet and the sample thus collected was utilized for identification of anti-inflammatory properties in various animal models.

Then the comparative assessment of various herbs with cow urine in terms of physical stability, organoleptic parameters etc and for chemical instability, samples was used to record the Fourier-Transform Infrared FT-IR spectrum.

Then the final stable extract was incorporated in the nano fibre patches with the help of suitable polymer through the electrospinning machine.

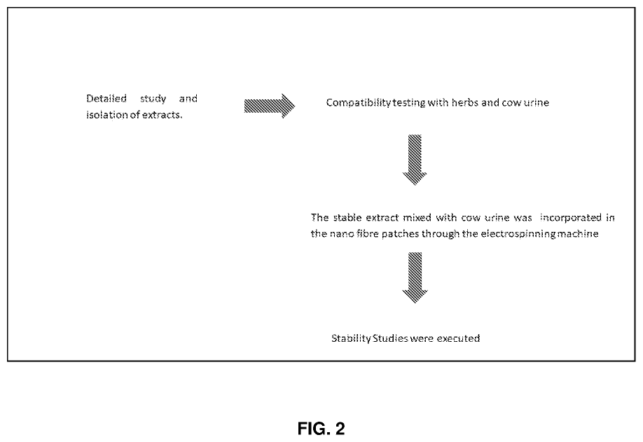

For developing this bioactive dressing as shown in FIG. 2, the dry powder obtained from plant sources was extracted with cow urine at 40-45 deg C. by maceration process for 72 hrs. Later on, the cow urine extract was filtered and the filtrate was concentrated to a semi solid mass by using vacuum distillation apparatus. The extract so obtained was used for the phytochemical and pharmacological investigations. After preparing the present invention, the pre-clinical studies on animal models by examining the wound healing process on Control, Treated and Non-Treated groups for better evaluation of the results.

The bioactive dressing was developed using electro spin machine. The we have evaluated the wound healing property of cow urine using excision wound model in rats with the rate of wound contraction as a parameter.

A nanofibre patch was developed as they often show poor adhesion to the base support. The plasma-treated cotton gauzes were used as the support fabric after electrospinning chitosan nanofibers, where nanofibers showed better adhesion, a low rate of degradation and moisture vapor transport. The use of cow urine and herbal extracts helped in fastening the wound healing process.

The cow urine helps in augmentation of B, T-lymphocyte blastogenesis and also increases the release of IL-1 & 2 (interleukin). Which is an important component in the wound healing process.

The cow urine herbal nanofibers of the dressing significantly enhance the collagen content and granulation tissue formation in wounds. Thus, it can be useful in accelerating wound healing in patients because of its property of enhancing granulation tissue formation.

Following Table 1 represents histological changes with comparison of the bioactive dressing with standard bandage and antiseptic cream used for minor wounds.

| | In control Group with | In Test group where we |
|---|---|---|
| | Standard bandage with | have applied our Nano |
| | cream | fiber Dressing |
| Neutrophils | 1.5 ± 0.65 | 1.8 ± 0.15 |
| Macrophages | 0.9 ± 0.2 | 1.4 ± 0.2 |
| Fibroblasts | 1.1 ± 0.3 | 2.1 ± 0.15 |

N = 12 in each group, where values are represented as mean ± SEMs (Dunns multiple comparison test

While the foregoing describes various embodiments of the invention, other

and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

Thus, the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A herbal bioactive dressing for healing wounds, the dressing comprising an outer layer comprising a matrix of nanofibre formed by mixing cow urine with extracts obtained from Aloe Vera, Calandula, Curcumin, Sandal wood in same amount and a self-adhesive inner layer.

2. The dressing of claim 1, wherein the dressing incorporated into the nanofiber patches through electrospinning.

3. The dressing of claim 1, wherein the matrix is non-absorptive and non-dissolving.

4. The dressing of claim 1, wherein the extract is in dry powder form.

5. A method for preparation of a herbal bioactive dressing for healing wounds, the method comprising:
isolating extracts from a plurality of herbs comprising Aloe Vera, Calandula, Curcumin, Sandal wood;
collecting cow urine at different interval from five different indigenous breeds of cows;
performing comparative assessment of the herbs with cow urine in terms of physical stability, organoleptic parameters and chemical instability;
mixing the cow urine with the extracts at 40-45 deg C. by maceration process for 72 hours to form a cow urine extract;
filtering the cow urine extract to obtain the filtrate;
concentrating the filtrate to a semi solid mass by using vacuum distillation apparatus; and
incorporating the semi solid mass into a nanofiber patch.

6. The method of claim 5, wherein the extract is dry powder.

7. The method of claim 5, wherein the maceration process involves pouring at least 25 grams of each of the herbs into at least 150 cc absolute ethanol, followed by placing the mixture in a shaking incubator at 30 deg C and at 100 rpm for 48 hours.

8. The method of claim 7, wherein the maceration process involves evaporating the ethanolic extract using rotary evaporator for at least 25 min to obtain pasty extract.
